(19) United States
(12) Patent Application Publication
(10) Pub. No.: US 2024/0159728 A1
(43) Pub. Date: May 16, 2024

(54) CARBON ISOTOPE RATIOS TO IDENTIFY SOURCE ROCKS

(71) Applicant: Saudi Arabian Oil Company, Dhahran (SA)

(72) Inventor: Feng Hu Lu, Dhahran (SA)

(21) Appl. No.: 18/464,611

(22) Filed: Sep. 11, 2023

Related U.S. Application Data
(60) Provisional application No. 63/425,115, filed on Nov. 14, 2022.

(51) Int. Cl. G01N 33/24; G01N 15/08

(52) U.S. Cl. CPC G01N 33/24; G01N 15/08

(57) ABSTRACT

The disclosure relates to methods for identifying desirable (e.g., the best) depth intervals in a reservoir using carbon isotope ratio information. The desirable depth intervals in the reservoir correspond to regions in the reservoir with source rocks and where natural gas is stored.

### CROSS-REFERENCE TO RELATED PATENT APPLICATIONS

This application claims the benefit of U.S. Provisional Application No. 63/425,115, filed Nov. 14, 2022, the contents of which are incorporated by reference herein.

### FIELD

The disclosure relates to methods for identifying desirable (e.g., the best) depth intervals in a reservoir using carbon isotope ratio information. The desirable depth intervals in the reservoir correspond to regions in the reservoir with source rocks and where natural gas is stored.

### BACKGROUND

Natural gas contains CO₂ that was originally produced along with hydrocarbon gas compounds (e.g., methane) from the cracking of kerogen. CO₂ from other sources can also be present.

### SUMMARY

The disclosure relates to methods for identifying desirable (e.g., the best) depth intervals in a reservoir using carbon isotope ratio information. The desirable depth intervals in the reservoir correspond to regions in the reservoir with source rocks and where natural gas is stored.

The methods can allow for the discovery of source rocks and areas which natural gas is stored. The methods can allow for the identification of the location of source rocks relatively quickly and/or inexpensively compared to certain other methods of identifying source rocks.

The methods can allow for the observation of interval(s) with source rocks and areas in which natural gas is stored in a reservoir, which can be relatively hard to identify using certain geophysical methods. Additionally, the interval(s) can be determined and correlated from one studied well to the adjacent wells in the same gas field.

Without wishing to be bound by theory, it is believed that the methods of the disclosure can identify source rocks with fewer measurements of multiple wells and/or outcrops relative to certain other methods of identifying deposits or organic matter. Furthermore, the methods can reduce time and/or costs associated with the measurement of samples to determine the location of source rocks relative to certain other methods of identifying the location of source rocks as measurements from various wells and/or outcrops can be reduced.

In a first aspect, the disclosure provides a method of determining a depth of source rocks within a carbonate formation, the method including: measuring a carbon isotope ratio of carbonate samples taken from the carbonate formation at a plurality of depths within the carbonate formation; measuring a carbon isotope ratio of carbon dioxide in a gas produced from the carbonate formation; generating a data set including the measured carbon isotope ratio of the carbonate versus depth of the carbonate formation; and using the measured carbon isotope ratio of carbon dioxide and the data set to determine a depth of the source rocks in the carbonate formation.

In some embodiments, using the measured carbon isotope ratio of carbon dioxide and the data set to determine the depth of the source rocks in the carbonate formation includes determining a depth at which a difference between the measured carbon isotope ratio of the carbonate formation and the carbon dioxide is minimized.

In some embodiments, the method further includes measuring a porosity of the carbonate samples taken from the carbonate formation; and the data set further includes the measured porosity versus depth of the carbonate formation.

In some embodiments, the carbon isotope ratio of carbon dioxide in a gas produced from the carbonate formation is 0‰ to 4‰.

In some embodiments, a sampling interval of carbonate samples taken from the carbonate formation is 0.3 meters to 10 meters.

In some embodiments, the difference between the measured carbon isotope ratio of the carbonate formation and the carbon dioxide is less than 2‰. In some embodiments, the difference between the measured carbon isotope ratio of the carbonate formation and the carbon dioxide is less than 1‰.

In some embodiments, the method further includes measuring a porosity of the carbonate samples taken from the carbonate formation; and the data set further includes the measured porosity versus depth of the carbonate formation.

In some embodiments, the carbon isotope ratio of carbon dioxide in a gas produced from the carbonate formation is 0‰ to 4‰.

In some embodiments, a sampling interval of carbonate samples taken from the carbonate formation is 0.3 meters to 10 meters.

In some embodiments, the carbon isotope ratio of carbon dioxide in a gas produced from the carbonate formation is 0‰ to 4‰.

In some embodiments, a sampling interval of carbonate samples taken from the carbonate formation is 0.3 meters to 10 meters.

In some embodiments, the carbonate samples include cored carbonate rocks. In some embodiments, the carbonate samples are powdered samples.

In some embodiments, the carbonate samples include outcrop carbonate rocks. In some embodiments, the carbonate samples are powdered samples.

In some embodiments, the carbonate samples are powdered samples.

In some embodiments, the method further includes, producing a hydrocarbon from the depth determined using the data set.

In a second aspect, the disclosure provides one or more machine-readable hardware storage devices including instructions that are executable by one or more processing devices to perform operations including a method of the disclosure.

In a third aspect, the disclosure provides a system including one or more processing devices; and one or more machine-readable hardware storage devices including instructions that are executable by the one or more processing devices to perform operations including a method of the disclosure.

### BRIEF DESCRIPTION OF THE FIGURES

FIG. 1 depicts a plot of carbon isotope ratios of carbonate samples versus depth in a reservoir and the carbon isotope ratio of CO₂ from a gas produced from the reservoir.

FIG. 2 depicts a plot of carbon and oxygen isotope ratios of carbonate samples versus depth in a reservoir and the carbon isotope ratio of CO₂ from a gas produced from the reservoir.

FIG. 3 depicts a flowchart for a method.

FIG. 4 depicts a schematic of hydrocarbon production operations.

### DETAILED DESCRIPTION

### Methods Using Carbon Isotope Ratios From CO₂

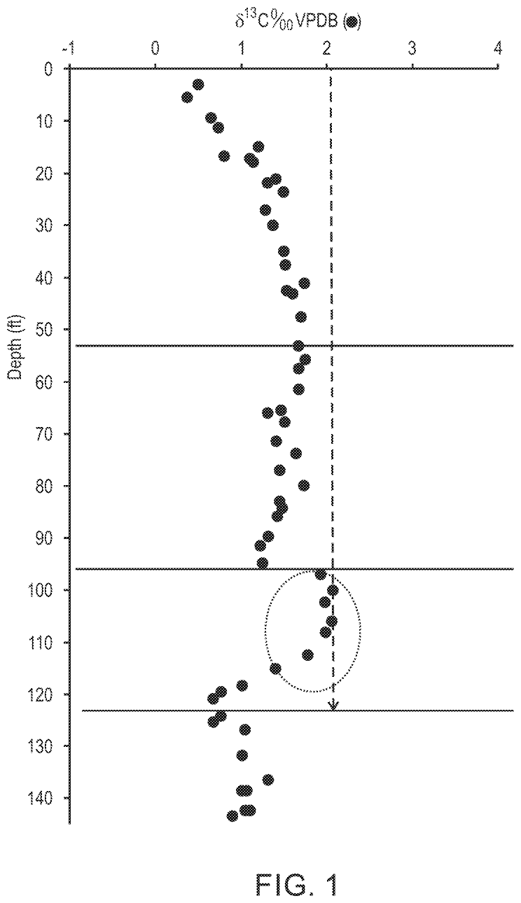

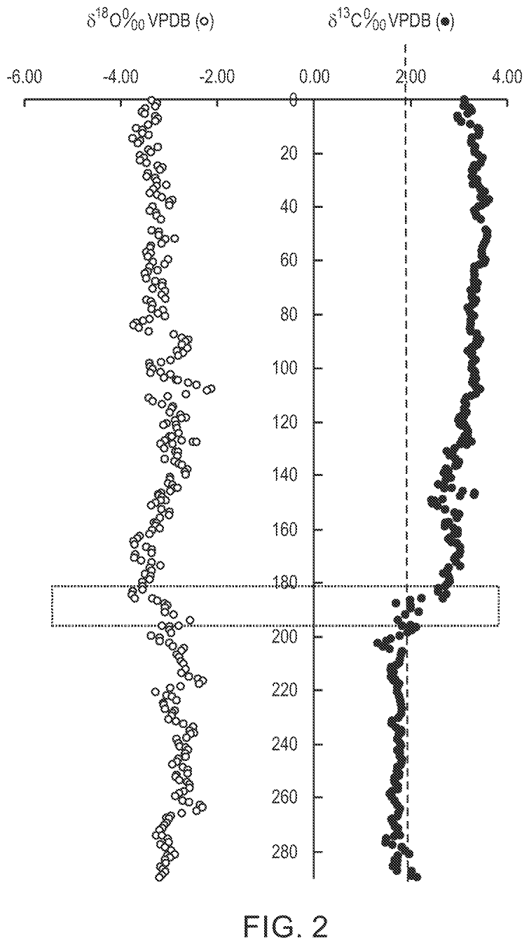

FIG. 1 depicts a plot of the carbon isotope ratio versus depth in a reservoir (δ¹³C‰ VPDB) and FIG. 2 depicts a plot of carbon and oxygen isotope ratios of carbonate samples (δ¹³C‰ VPDB and δ¹⁸O‰ VPDB, respectively) versus depth in a different reservoir. The carbon and oxygen isotope ratio values were obtained from carbonate samples taken from the reservoir at different depths from the surface. The minimum depth shown was set to 0. Additionally, for the reservoirs in FIGS. 1 and 2, a gas was produced from the reservoir and the carbon isotope ratio of CO₂ in the produced gas was measured. The carbon isotope ratio of the CO₂ from the produced gas is plotted as a vertical dashed line allowing for comparison to the carbon isotope ratio of the carbonate samples. Details regarding the construction of FIGS. 1 and 2 are provided below.

Without wishing to be bound by theory, it is believed that a depth at which the value of the carbon isotope ratio of the carbonate samples matches the value of the carbon isotope ratio of the CO₂ from the produced gas corresponds to a region with source rocks and the area which natural gas is stored. This region is indicated by a dotted oval in FIG. 1 and a dotted rectangle in FIG. 2. Without wishing to be bound by theory, it is believed that the carbon isotope ratio measured from CO₂ in the produced gas sample is an average of gas produced from the reservoir but dominated by the values in a region with source rocks and the area where natural gas is stored.

Without wishing to be bound by theory, it is believed that natural gas contains a variety of CO₂ that was produced with hydrocarbons (e.g., methane) from the cracking of kerogen which typically has a negative carbon isotope ratio due to its organic origin (e.g., <−10‰). Additionally, CO₂ and natural gas would remain in the reservoir where it was produced from the source rocks. Certain carbonate-containing reservoirs (e.g., marine carbonates) typically have positive carbon isotope ratios (e.g., 0 to 5‰ or higher). Over time, the CO₂ and carbonate rock interact (e.g., via water as a mediator) and carbon isotope exchange occurs.

Carbon isotope ratios of CO₂ in gas can change, and the change generally depends on geological conditions in which CO₂ in the gas and carbonate rocks can interact. For example, carbonate dissolution can cause the release CO₂, which can mix with the CO₂ generated from kerogen cracking. CO₂ can also react with water to form carbonate. Geological conditions can determine which of the reactions is favored. A relatively large amount of carbonate rocks are present, so the dissolution of carbonates to produce CO₂ is eventually favored, meaning carbon isotopes from carbonate will dominate kerogen-gas CO₂ carbon isotopes.

If a sufficient amount of CO₂ reacts with calcium to precipitate carbonate cements in the host rocks, the carbonate cements would bear the typical negative carbon isotopes (e.g., <−10‰, −20‰ to −10‰). In contrast, if an amount of CO₂ generated from kerogen is relatively small and the carbonates dissolve and release CO₂, then a much larger amount of CO₂ derived from dissolved carbonates will be present and the total CO₂ in the carbonate reservoir would have the carbon isotopes of carbonate host rocks (e.g., 0‰ to 5‰). The amount of kerogen-sourced CO₂ may be relatively small (e.g., 1%) of the total amount of CO₂ due to the limited amount of oxygen in kerogen, whereas carbonate rocks can provide an essentially unlimited supply of CO₂. During diagenesis/catagenesis and petroleum generation, formation water is often acidic, resulting in carbonate dissolution and CO₂ release from the host rocks. Thus, CO₂ released from the host rocks can dominate the CO₂ generated from kerogen. Therefore, with sufficient time, the CO₂ in the reservoir gas would have essentially the same carbon isotopes as the host carbonates. The carbonate bed or interval bearing the same carbon isotopes to that of CO₂ in the gas reservoir is where natural gas is stored, which can be determined from the plots described above. The carbonate interval may be the source rocks (kerogen for the gas) and may not be the exact the source bed but a reservoir bed in the same formation.

Without wishing to be bound by theory, it is believed that in some embodiments, CO₂ from carbonates can mix with the CO₂ from the produced gas and alter the carbon isotope ratio.

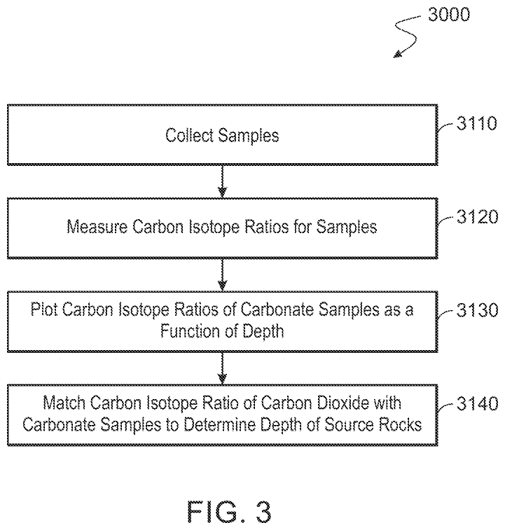

FIG. 3 depicts a flowchart for a method 3000 for identifying the depth of source rocks and areas which natural gas is stored.

In step 3110, samples are collected from a carbonate formation. The samples include i) CO₂ from a natural gas sample produced from the carbonate formation and ii) carbonate samples (e.g., carbonates from cored samples) collected at a plurality of depths.

As noted above, natural gas is produced from the carbonate formation. In some embodiments, the natural gas sample is produced from an exploration well. For the CO₂ from the produced gas, one or more measurements (e.g., at least two, at least three, at least four, at least five) may be performed depending on how the sample is collected.

In step 3120, the carbon isotope ratios are measured in the carbonate samples and the CO₂ from the produced gas. In step 2120, the carbon and oxygen isotope ratios in the carbonate samples are measured. In general, the carbon and oxygen isotope ratios can be determined using any suitable method. In certain embodiments, carbon and oxygen isotope ratios are determined using isotope ratio mass spectrometry with appropriate standards.

The carbon isotope ratio can be calculated using the equation:

$${δ}^{13} C = ( \frac{{( {}^{13} C / {}^{12} C )}_{sample}}{{( {}^{13} C / {}^{12} C )}_{standard}} - 1 ) \times 1000$$

Similarly, the oxygen isotope ratio can be calculated using the equation:

$${δ}^{18} O = ( \frac{{( {}^{18} O / {}^{16} O )}_{sample}}{{( {}^{18} O / {}^{16} O )}_{standard}} - 1 ) \times 1000$$

In certain embodiments, in the step 3120, oxygen isotope ratios are also measured in the CO₂ from the produced gas and/or the carbonate samples.

In step 3130, the carbon isotope ratio of the carbonate samples versus depth is plotted. The carbon isotope ratio of the produced gas sample is also plotted. Such plots are depicted in FIGS. 1 and 2.

In step 3140, using the plot from step 3130, the depth at which the carbon isotope ratio of CO₂ in the produced gas matches the carbon isotope ratios of the carbonate samples is determined. As discussed above, this depth corresponds to the location of source rocks and area where natural gas is stored.

In certain embodiments, the difference between the carbon isotope ratio in the CO₂ from the produced gas and the carbonate formation is at most 2 (e.g., at most 1.5, at most 1, at most 0.5, at most 0.1, at most 0.05, at most 0.01) ‰.

In some embodiments, the carbon isotope ratio of the CO₂ from the produced gas is at least 0 (e.g., at least 0.5, at least 1, at least 1.5, at least 2, at least 2.5, at least 3, at least 3.5) ‰ and/or at most 4 (e.g., at most 3.5, at most 3, at most 2.5, at most 2, at most 1.5, at most 1, at most 0.5) ‰.

The carbonate samples collected in step 3110 and measured in step 3120 can include cored carbonate rocks or outcrop carbonate rocks. Powder samples are then obtained (e.g., using a drill to drill into a sample of the cored carbonate rock or the outcrop carbonate rock). In some embodiments, the samples are from core samples. Without wishing to be bound by theory, it is believed that, when making the isotope measurements, more representative results are achieved using powdered rock samples, and that, as a result, in some embodiments, cements, clastic fragments and fossils should be avoided when making the isotope measurements.

Generally, the depths of the reservoir that are investigated depends on local geological conditions. In general, the interval between samples may be selected based on the depositional environment of the source rock. In some embodiments, the interval between samples is at least 0.3 (e.g., at least 0.5, at least 1, at least 1.5, at least 2, at least 2.5, at least 3, at least 3.5, at least 4, at least 4.5, at least 5, at least 5.5, at least 6, at least 6.5, at least 7, at least 7.5, at least 8, at least 8.5, at least 9, at least 9.5) meters and/or at most 10 (e.g., at most 9.5, at most 9, at most 8.5, at most 8, at most 7.5, at most 7, at most 6.5, at most 6, at most 5.5, at most 5, at most 4.5, at most 4, at most 3.5, at most 3, at most 2.5, at most 2, at most 1.5, at most 1, at most 0.5) meters. In some embodiments, the interval between samples is 0.3 meters. In some embodiments, the interval between samples is 0.9 meters. In general, the interval between samples 1 is meter or less. In general, the sampling interval depends on targeted thickness.

Generally, the sampling interval depends on presence of changes in rocks, such as the changes in fossils, lithology, and rock texture. A sample may be taken at the spot with a change, and samples within this region can be taken at a sampling interval of 0.1 m to 10 m, taking into consideration the total studied thickness.

Without wishing to be bound by theory, it is believed that increasing the total number of samples (denser sampling) over the reservoir can yield higher resolution. If the targeted reservoir is relatively large and the location of the source rocks is not known, a first scan with a relatively large sampling interval can be used to reduce work and cost. After the initial scan, a detailed sampling using a smaller sampling interval in a portion of the reservoir section may be conducted.

Without wishing to be bound by theory, it is believed that the interval can be correlated to the adjacent areas in the same field.

Data obtained from the measurements, such as carbon isotope ratio information versus depth and carbon isotope ratio of the CO₂ from produced gas, can be in the form of a data set. The data set can be used to generate the plots and/or identify the desirable intervals.

In general, the depth at which the carbon isotope ratio of CO₂ in the produced gas matches the carbon isotope ratios of the carbonate samples can be determined by visual inspection of the plots (e.g., FIGS. 1 and 2) and/or using an appropriate algorithm.

### Computational Operations

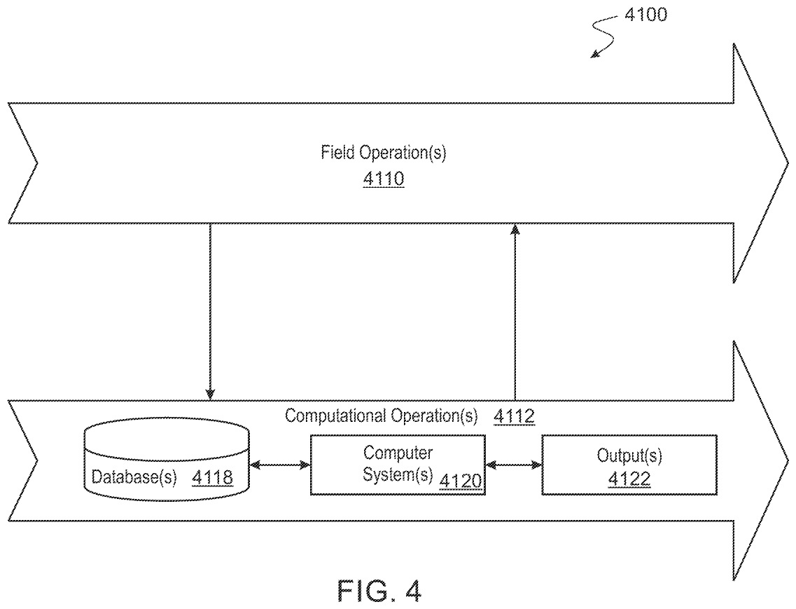

FIG. 4 illustrates hydrocarbon production operations 4100 that include both one or more field operations 4110 and one or more computational operations 4112, which exchange information and control exploration for the production of hydrocarbons. In some implementations, outputs of techniques of the present disclosure can be performed before, during, or in combination with the hydrocarbon production operations 4100, specifically, for example, either as field operations 4110 or computational operations 4112, or both. Examples of field operations 4110 include forming/drilling a wellbore, hydraulic fracturing, producing through the wellbore, injecting fluids (such as water) through the wellbore, to name a few. In some implementations, methods of the present disclosure can trigger or control the field operations 4110. For example, the methods of the present disclosure can generate data from hardware/software including sensors and physical data gathering equipment (e.g., seismic sensors, well logging tools, flow meters, and temperature and pressure sensors). The methods of the present disclosure can include transmitting the data from the hardware/software to the field operations 4110 and responsively triggering the field operations 4110 including, for example, generating plans and signals that provide feedback to and control physical components of the field operations 4110. Alternatively or in addition, the field operations 4110 can trigger the methods of the present disclosure. For example, implementing physical components (including, for example, hardware, such as sensors) deployed in the field operations 4110 can generate plans and signals that can be provided as input or feedback (or both) to the methods of the present disclosure.

Examples of computational operations 4112 include one or more computer systems 4120 that include one or more processors and computer-readable media (e.g., non-transitory computer-readable media) operatively coupled to the one or more processors to execute computer operations to perform the methods of the present disclosure. The computational operations 4112 can be implemented using one or more databases 4118, which store data received from the field operations 4110 and/or generated internally within the computational operations 4112 (e.g., by implementing the methods of the present disclosure) or both. For example, the one or more computer systems 4120 process inputs from the field operations 4110 to assess conditions in the physical world, the outputs of which are stored in the databases 4118. For example, seismic sensors of the field operations 4110 can be used to perform a seismic survey to map subterranean features, such as facies and faults. In performing a seismic survey, seismic sources (e.g., seismic vibrators or explosions) generate seismic waves that propagate in the earth and seismic receivers (e.g., geophones) measure reflections generated as the seismic waves interact with boundaries between layers of a subsurface formation. The source and received signals are provided to the computational operations 4112 where they are stored in the databases 4118 and analyzed by the one or more computer systems 4120.

In some implementations, one or more outputs 4122 generated by the one or more computer systems 4120 can be provided as feedback/input to the field operations 4110 (either as direct input or stored in the databases 4118). The field operations 4110 can use the feedback/input to control physical components used to perform the field operations 4110 in the real world.

For example, the computational operations 4112 can process the seismic data to generate three-dimensional (3D) maps of the subsurface formation. The computational operations 4112 can use these 3D maps to provide plans for locating and drilling exploratory wells. In some operations, the exploratory wells are drilled using logging-while-drilling (LWD) techniques which incorporate logging tools into the drill string. LWD techniques can enable the computational operations 4112 to process new information about the formation and control the drilling to adjust to the observed conditions in real-time.

The one or more computer systems 4120 can update the 3D maps of the subsurface formation as information from one exploration well is received and the computational operations 4112 can adjust the location of the next exploration well based on the updated 3D maps. Similarly, the data received from production operations can be used by the computational operations 4112 to control components of the production operations. For example, production well and pipeline data can be analyzed to predict slugging in pipelines leading to a refinery and the computational operations 4112 can control machine operated valves upstream of the refinery to reduce the likelihood of plant disruptions that run the risk of taking the plant offline.

In some implementations of the computational operations 4112, customized user interfaces can present intermediate or final results of the above-described processes to a user. Information can be presented in one or more textual, tabular, or graphical formats, such as through a dashboard. The information can be presented at one or more on-site locations (such as at an oil well or other facility), on the Internet (such as on a webpage), on a mobile application (or app), or at a central processing facility.

The presented information can include feedback, such as changes in parameters or processing inputs, that the user can select to improve a production environment, such as in the exploration, production, and/or testing of petrochemical processes or facilities. For example, the feedback can include parameters that, when selected by the user, can cause a change to, or an improvement in, drilling parameters (including drill bit speed and direction) or overall production of a gas or oil well. The feedback, when implemented by the user, can improve the speed and accuracy of calculations, streamline processes, improve models, and solve problems related to efficiency, performance, safety, reliability, costs, downtime, and the need for human interaction.

In some implementations, the feedback can be implemented in real-time, such as to provide an immediate or near-immediate change in operations or in a model. The term real-time (or similar terms as understood by one of ordinary skill in the art) means that an action and a response are temporally proximate such that an individual perceives the action and the response occurring substantially simultaneously. For example, the time difference for a response to display (or for an initiation of a display) of data following the individual's action to access the data can be less than 1 millisecond (ms), less than 1 second (s), or less than 5 s. While the requested data need not be displayed (or initiated for display) instantaneously, it is displayed (or initiated for display) without any intentional delay, taking into account processing limitations of a described computing system and time required to, for example, gather, accurately measure, analyze, process, store, or transmit the data.

Events can include readings or measurements captured by downhole equipment such as sensors, pumps, bottom hole assemblies, or other equipment. The readings or measurements can be analyzed at the surface, such as by using applications that can include modeling applications and machine learning. The analysis can be used to generate changes to settings of downhole equipment, such as drilling equipment. In some implementations, values of parameters or other variables that are determined can be used automatically (such as through using rules) to implement changes in oil or gas well exploration, production/drilling, or testing. For example, outputs of the present disclosure can be used as inputs to other equipment and/or systems at a facility. This can be especially useful for systems or various pieces of equipment that are located several meters or several miles apart, or are located in different countries or other jurisdictions.

### Additional Measurements

In certain embodiments, a method of the disclosure can further include studying the general mineral composition using an appropriate technique such as XRD or staining. For example, staining can be used to differentiate dolomite from aragonite and calcite. Alizarin red S and potassium ferricyanide dissolved in a dilute hydrochloric acid (1%) solution can stain the more reactive calcite and aragonite red while the less reactive dolomite remains unstained. In some embodiments, slabs or thin sections made from representative lithology are placed in the stain solution at 25° C. for 1-2 minutes. XRD can also provide the mineral composition. Without wishing to be bound by theory, it is believed that studying the general mineral composition can provide relatively quick information regarding mineral composition and depositional and diagenetic environments.

Without wishing to be bound by theory, it is believed that the area where natural gas is stored is expected to contain relatively high porosity, as diagenesis and catagenesis would cause dissolution of the host carbonate rocks due to the formation of organic acid, resulting in the development of porosity and the release of CO₂ within this interval. Additionally, the cracking process of carrageenan can generate porosity as pore space is created by the loss of carriage and matter. Petrographic studies and/or other well logging data can be utilized to confirm high porosity.

The porosity can be determined using fluid/gas injection, an image analysis, a well logging technique, a seismic analysis, and/or well testing. In certain embodiments, a petrography study is performed to evaluate segmentation and porosity to identify intervals with relatively high porosity. In certain embodiments, well logging data is used to determine porosity.

### EXAMPLE

Cored carbonate samples were collected from an exploration/production well in a studied field/basin. A dental drill was used to drill into the slab in a matrix area to obtain powder samples. To obtain representative samples, cements, clastic fragments and fossils were avoided.

Oxygen and carbon isotope ratios were analyzed in the carbonate samples using isotope ratio mass spectrometry. If the samples contained large grains and were not fine enough for the measurements, a mortar and pestle were used to grind the grains to a homogeneous powder. Powered samples were placed in a small vial (10 mL). The vials with samples but without caps were placed in a 50° C. oven overnight to dry the powder samples. The samples were subsequently analyzed by isotope ratio mass spectrometry (IRMS) for carbon and oxygen isotope analyses. A Thermo-Fisher IRMS and gasbench instrument was used for sample processing. Each of the samples was weighed to about 300 μg using weighing paper and the powder was transferred into a reaction vial. Powdered carbonate samples along with a set of standards (international and local carbonate standards with known O and C isotope values) were also weighed and placed into vials. Each vial was capped with a septum. An automated sampler vacuumed the vials before 4 drops (400 μg) concentrated H₃PO₄ acid was to produce CO₂ gas. The CO₂ was transferred to the isotope ratio mass spectrometer, where each of the standard and rock samples were measured with a C-isotope ratio and O-isotope ratio simultaneously. The standards were used to calibrate the C and O-isotopes of rock samples, and to check accuracy and precision of analysis in each set.

The carbon and optionally oxygen isotopes ratios were plotted versus depth of the reservoir using Excel, as shown in FIGS. 1 and 2. The carbon and oxygen isotope ratios varied versus depth.

Carbon and oxygen isotope ratios of CO₂ from a natural gas sample from the same well were measured. The gas sample was usually collected from a long interval within the targeted reservoir. The carbon isotope ratio of the CO₂ was matched with the carbon isotope ratio of the carbonate formation versus depth. The depth at which the two carbon isotope ratios match was identified, which corresponds to the source rocks and the interval where natural gas is stored.

FIG. 1 shows carbon isotope ratios versus depth from one well in a field. The carbon isotope ratio measured from CO₂ from the produced gas is shown as the vertical dashed line. In this well, the δ ¹³C for the CO₂ in the produced gas sample was 2‰. The δ ¹³C of the carbonates that was 2‰ and matched the δ ¹³C for the CO₂ in the produced gas from this well is indicated by the dashed oval. Therefore, this interval was determined as the best reservoir.

FIG. 2 shows the carbon and oxygen isotope ratios (black and white circles, respectively) from another well/field. The carbon isotope ratio measured from CO₂ from the produced gas is shown as the vertical dashed line. The interval highlighted with the dotted rectangle had a carbon isotope ratio corresponding to that of the CO₂ from the produced gas sample. This interval is the interval with source rocks and where natural gas is stored in this well/field.

The intervals identified above were confirmed by mud gas profiling which indicated the highest gas levels occurred within these intervals. These intervals can be identified and correlated the adjacent areas in the same field.

### Embodiments

1. A method of determining a depth of source rocks within a carbonate formation, the method including:
measuring a carbon isotope ratio of carbonate samples taken from the carbonate formation at a plurality of depths within the carbonate formation;
measuring a carbon isotope ratio of carbon dioxide in a gas produced from the carbonate formation;
generating a data set including the measured carbon isotope ratio of the carbonate versus depth of the carbonate formation; and
using the measured carbon isotope ratio of carbon dioxide and the data set to determine a depth of the source rocks in the carbonate formation.

2. The method of embodiment 1, wherein using the measured carbon isotope ratio of carbon dioxide and the data set to determine the depth of the source rocks in the carbonate formation includes:
determining a depth at which a difference between the measured carbon isotope ratio of the carbonate formation and the carbon dioxide is minimized.

3. The method of embodiment 1 or 2, wherein:
the method further includes measuring a porosity of the carbonate samples taken from the carbonate formation; and
the data set further includes the measured porosity versus depth of the carbonate formation.

4. The method of any one of embodiments 1-3, wherein the carbon isotope ratio of carbon dioxide in a gas produced from the carbonate formation is 0‰ to 4‰.

5. The method of any one of embodiments 1-4, wherein a sampling interval of carbonate samples taken from the carbonate formation is 0.3 meters to 10 meters.

6. The method of any one of embodiments 1-5, wherein the difference between the measured carbon isotope ratio of the carbonate formation and the carbon dioxide is less than 2‰.

7. The method of any one of embodiments 1-5, wherein the difference between the measured carbon isotope ratio of the carbonate formation and the carbon dioxide is less than 1‰.

8. The method of any one of embodiments 1-7, wherein:
the method further includes measuring a porosity of the carbonate samples taken from the carbonate formation; and
the data set further includes the measured porosity versus depth of the carbonate formation.

9. The method of embodiment 8, wherein the carbon isotope ratio of carbon dioxide in a gas produced from the carbonate formation is 0‰ to 4‰.

10. The method of embodiment 8, wherein a sampling interval of carbonate samples taken from the carbonate formation is 0.3 meters to 10 meters.

11. The method of embodiment 1, wherein the carbon isotope ratio of carbon dioxide in a gas produced from the carbonate formation is 0‰ to 4‰.

12. The method of embodiment 1, wherein a sampling interval of carbonate samples taken from the carbonate formation is 0.3 meters to 10 meters.

13. The method of any one of embodiments 1-12, wherein the carbonate samples include cored carbonate rocks.

14. The method of embodiment 13, wherein the carbonate samples are powdered samples.

15. The method of any one of embodiments 1-12, wherein the carbonate samples include outcrop carbonate rocks.

16. The method of embodiment 15, wherein the carbonate samples are powdered samples.

17. The method of any one of embodiments 1-12, wherein the carbonate samples are powdered samples.

18. The method of any one of embodiments 1-17, further including, producing a hydrocarbon from the depth determined using the data set.

19. One or more machine-readable hardware storage devices including instructions that are executable by one or more processing devices to perform operations including the method of any one of embodiments 1-18.

20. A system including:
one or more processing devices; and
one or more machine-readable hardware storage devices including instructions that are executable by the one or more processing devices to perform operations including the method of any one of embodiments 1-18.

what is claimed:

1. A method of determining a depth of source rocks within a carbonate formation, the method comprising:
measuring a carbon isotope ratio of carbonate samples taken from the carbonate formation at a plurality of depths within the carbonate formation;
measuring a carbon isotope ratio of carbon dioxide in a gas produced from the carbonate formation;
generating a data set comprising the measured carbon isotope ratio of the carbonate versus depth of the carbonate formation; and
using the measured carbon isotope ratio of carbon dioxide and the data set to determine a depth of the source rocks in the carbonate formation.

2. The method of claim 1, wherein using the measured carbon isotope ratio of carbon dioxide and the data set to determine the depth of the source rocks in the carbonate formation comprises:
determining a depth at which a difference between the measured carbon isotope ratio of the carbonate formation and the carbon dioxide is minimized.

3. The method of claim 1, wherein:
the method further comprises measuring a porosity of the carbonate samples taken from the carbonate formation; and
the data set further comprises the measured porosity versus depth of the carbonate formation.

4. The method of claim 1, wherein the carbon isotope ratio of carbon dioxide in a gas produced from the carbonate formation is 0‰ to 4‰.

5. The method of claim 1, wherein a sampling interval of carbonate samples taken from the carbonate formation is 0.3 meters to 10 meters.

6. The method of claim 1, wherein the difference between the measured carbon isotope ratio of the carbonate formation and the carbon dioxide is less than 2‰.

7. The method of claim 1, wherein the difference between the measured carbon isotope ratio of the carbonate formation and the carbon dioxide is less than 1‰.

8. The method of claim 1, wherein:
the method further comprises measuring a porosity of the carbonate samples taken from the carbonate formation; and
the data set further comprises the measured porosity versus depth of the carbonate formation.

9. The method of claim 8, wherein the carbon isotope ratio of carbon dioxide in a gas produced from the carbonate formation is 0‰ to 4‰.

10. The method of claim 8, wherein a sampling interval of carbonate samples taken from the carbonate formation is 0.3 meters to 10 meters.

11. The method of claim 1, wherein the carbon isotope ratio of carbon dioxide in a gas produced from the carbonate formation is 0‰ to 4‰.

12. The method of claim 1, wherein a sampling interval of carbonate samples taken from the carbonate formation is 0.3 meters to 10 meters.

13. The method of claim 1, wherein the carbonate samples comprise cored carbonate rocks.

14. The method of claim 13, wherein the carbonate samples are powdered samples.

15. The method of claim 1, wherein the carbonate samples comprise outcrop carbonate rocks.

16. The method of claim 15, wherein the carbonate samples are powdered samples.

17. The method of claim 1, wherein the carbonate samples are powdered samples.

18. The method of claim 1, further comprising, producing a hydrocarbon from the depth determined using the data set.

19. One or more machine-readable hardware storage devices comprising instructions that are executable by one or more processing devices to perform operations comprising the method of claim 1.

20. A system comprising:
one or more processing devices; and
one or more machine-readable hardware storage devices comprising instructions that are executable by the one or more processing devices to perform operations comprising the method of claim 1.
